(19) United States
(12) Patent Application Publication
(10) Pub. No.: US 2010/0068194 A1
(43) Pub. Date: Mar. 18, 2010

(54) COMPOSITION FOR IN VIVO TRANSPLANTATION FOR TREATMENT OF HUMAN CERVICAL CANCER COMPRISING MONONUCLEAR CELLS DERIVED FROM UMBILICAL CORD BLOOD

(76) Inventor: Dong-Ku Kim, Seoul (KR)

Correspondence Address: THE NATH LAW GROUP, 112 South West Street, Alexandria, VA, 22314, US

(21) Appl. No.: 12/312,026

(22) PCT Filed: Oct. 11, 2007
(86) PCT No.: PCT/KR2007/004962

§ 371 Date: Apr. 23, 2009

(30) Foreign Application Priority Data
Oct. 24, 2006 (KR) 10-2006-0103627

(51) Int. Cl. A61K 45/00; A61P 35/00

(52) U.S. Cl. 424/93.71

(57) ABSTRACT

Provided is a composition for in vivo transplantation for the treatment of human cervical cancer, comprising mononuclear cells derived from umbilical cord blood and a pharmaceutically acceptable carrier. When the umbilical cord blood-derived mononuclear cells are transplanted in vivo, cervical cancer can be effectively treated. In particular, the mononuclear cells derived from the umbilical cord blood retain high differentiation and proliferation abilities and exhibit very low graft-versus-host (GVH) reactions which are side effects caused by transplantation, and thus, can be transplanted to many patients.

### TECHNICAL FIELD

The present invention relates to a composition for in vivo transplantation for the treatment of human cervical cancer, comprising mononuclear cells derived from umbilical cord blood.

### BACKGROUND ART

Cancer is the second leading cause of death in humans. Chemotherapy (administration of anticancer drugs), radiation therapy, and surgery have been mainly used for cancer treatment. Cancer can be treated at the early stage by using any one of the above methods or combination of the methods. However, for cancer which has already progressed to the terminal stage, which shows metastasis to other tissues through blood, or which shows a recurrence of cancer, the above methods have a very low therapeutic effect.

For most solid tumors, surgery is followed by chemotherapy and radiation therapy. However, since the cancers cannot be completely eliminated, the risk of recurrence is remarkably increased due to residual cancer cells. Recent research has revealed that cancer stem cells are present in various cancer tissues, and in order to fundamentally treat cancers, the cancer stem cells must be eliminated (Leukemia stem cells. Luo L, Han Z C. Int J Hematol. 2006. 84:123-127, Cancer stem cells—new and potentially important targets for the therapy of oral squamous cell carcinoma. Costea D E, Tsinkalovsky O, Vintermyr O K, Johannessen A C, Mackenzie I C. Oral Dis. 2006. 12:443-54. Cancer stem cells. Guo W, Lasky J L 3rd, Wu H. Pediatr Res. 2006. 59:59-64). However, markers specific for cancer have not yet been found, which makes the fundamental treatment of cancer difficult.

In order to solve this problem, various cancer treatment techniques have been developed. Among them, attempts have been mostly made to treat cancers using cancer-specific antibodies or immune cells (A new generation of monoclonal and recombinant antibodies against cell-adherent prostate specific membrane antigen for diagnostic and therapeutic targeting of prostate cancer. Elsasser-Beile U, Wolf P, Gierschner D, Buhler P, Schultze-Seemann W, Wetterauer U. Prostate. 2006. 66:1359-70).

With respect to cancer treatment using cancer-specific antibodies, activation of immune cells and their complements using antibodies against cell surface proteins which are expressed specifically in cancer cells and administration of toxin-linked antibodies into human bodies have been attempted.

With respect to cancer treatment using immune cells, techniques of eliminating cancer cells by cancer cell-specific cytotoxic T cells included in mass-cultured immune cells have been developed. That is, immune cells extracted from a blood sample provided by a cancer patient are mass-cultured in vitro and are then administered to cancer patients (Adoptive transfer of tumor-reactive Melan-A-specific CTL clones in melanoma patients is followed by increased frequencies of additional Melan-A-specific T cells. Vignard V, Lemercier B, Lim A, Pandolfino M C, Guilloux Y, Khammari A, Rabu C, Echasserieau K, Lang F, Gougeon M L, Dreno B, Jotereau F, Labarriere N. J Immunol. 2005, 175:4797-805).

In addition, cell therapy using dendritic cells has been developed. This is a method of eliminating cancer cells by stimulating the activation and immune responses of T cells in vivo, the method including isolating and culturing dendritic cells having an excellent antigen-presenting function that can present antigens specific to cancer cells, in vitro; adding a patient's cancer cell lysate or a cancer-specific antigen protein to the culture solution; and administering the mixture to a patient (Vaccination of Japanese patients with advanced melanoma with peptide, tumor lysate or both peptide and tumor lysate-pulsed mature, monocyte-derived dendritic cells. Nakai N, Asai J, Ueda E, Takenaka H, Katoh N, Kishimoto S. J Dermatol. 2006. 33:462-72. Clinical evaluation of dendritic cell vaccination for patients with recurrent glioma: results of a clinical phase I/II trial. Yamanaka R, Homma J, Yajima N, Tsuchiya N, Sano M, Kobayashi T, Yoshida S, Abe T, Narita M, Takahashi M, Tanaka R. Clin Cancer Res. 2005, 11:4160-7).

Recently, a method of treating cancer using a graft-versus-tumor (GVT) has been developed. According to this method, donor's blood samples having genetically different human leukocyte antigens (HLAs) are extracted, and T cells and natural killer (NK) cells of the blood samples are administered to patients with cancer to induce graft-versus-host (GVH) reactions, thereby eliminating residual cancer cells (A phase 1 trial of donor lymphocyte infusions expanded and activated ex vivo via CD3/CD28 costimulation. Porter D L, Levine B L, Bunin N, Stadtmauer E A, Luger S M, Goldstein S, Loren A, Phillips J, Nasta S, Perl A, Schuster S, Tsai D, Sohal A, Veloso E, Emerson S, June C H. Blood. 2006, 107:1325-31. Allogeneic hematopoietic stem cell transplantation for metastatic breast cancer. Bishop M R. Haematologica. 2004, 89:599-605). This method can induce higher immune responses than a method using autoimmune cells, and thus, is expected to be effective as a new cell therapy for the treatment of cancer.

Meanwhile, according to a conventional cancer treatment method using immune cells, i.e., a method of transplanting immune cells isolated from peripheral blood, it is difficult to continue cancer treatment for a long time due to the low proliferation ability of the transplanted immune cells. Moreover, according to a cancer treatment method using immune cells isolated from peripheral blood, due to the GVH reactions, cells derived from a patient are cultured in vitro, and are then transplanted to the patient. However, the cells cultured in vitro are cells in the terminal cell cycle stage, and thus, retain limited cell proliferation ability. Therefore, even though the cells are transplanted to a patient, it is difficult to expect the long-term therapeutic efficacy of immune cells.

### DISCLOSURE OF THE INVENTION

### Technical Problem

The present invention provides for improvement in a conventional cancer treatment method for the treatment of cancer, especially cervical cancer, using immune cells.

### Technical Solution

When mononuclear cells derived from umbilical cord blood which can be isolated without significantly affecting human bodies were applied to patients with cervical cancer, it was found that the mononuclear cells show good regenerative potential of hematopoietic cells and that even human leukocyte antigen (HLA)-mismatched patients exhibited very low graft-versus-host (GVH) reactions, thereby mononuclear cells derived from umbilical cord blood being effective for the treatment of cervical cancer.

Therefore, the present invention provides a composition for in vivo transplantation for the treatment of human cervical cancer, comprising mononuclear cells derived from umbilical cord blood.

### BRIEF DESCRIPTION OF THE DRAWINGS

FIG. 1 shows flow cytometry results for stem cells and immune cells in mononuclear cells derived from umbilical cord blood (a shows CD3+ T cells, b shows CD34+ hematopoietic stem cells, and c shows CD19+ B cells);

FIG. 2 shows cervical tumors formed in mice transplanted with Caski cells, mice co-transplanted with Caski cells and umbilical cord blood-derived mononuclear cells, and mice transplanted with umbilical cord blood-derived mononuclear cells 2-3 weeks after transplantation with Caski cells;

FIG. 3 shows cervical tumors extracted from mice transplanted with Caski cells, mice co-transplanted with Caski cells and umbilical cord blood-derived mononuclear cells, and mice transplanted with umbilical cord blood-derived mononuclear cells 2-3 weeks after transplantation with Caski cells;

FIG. 4 shows sizes of tumors extracted from mice transplanted with Caski cells, mice co-transplanted with Caski cells and umbilical cord blood-derived mononuclear cells, and mice transplanted with umbilical cord blood-derived mononuclear cells 2-3 weeks after transplantation with Caski cells;

FIG. 5 shows weights of tumors extracted from mice transplanted with Caski cells, mice co-transplanted with Caski cells and umbilical cord blood-derived mononuclear cells, and mice transplanted with umbilical cord blood-derived mononuclear cells 2-3 weeks after transplantation with Caski cells; and

FIG. 6 shows umbilical cord blood-derived hematopoietic and immune cells present in the peripheral blood (a, b) and the spleen tissues (c) of animal models (mice transplanted with human cervical cancer cells).

### BEST MODE FOR CARRYING OUT THE INVENTION

According to an aspect of the present invention, there is provided a composition for in vivo transplantation for the treatment of human cervical cancer, comprising mononuclear cells derived from umbilical cord blood and a pharmaceutically acceptable carrier. The umbilical cord blood-derived mononuclear cells may include CD34+ hematopoietic stem cells, CD3+ T cells and CD19+ B cells.

Mononuclear cells derived from umbilical cord blood according to the present invention are present as immature cells and retain remarkable differentiation and proliferation abilities. Cells obtained by in vitro culture of immune cells derived from peripheral blood are cells in the terminal cell cycle stage, and thus, retain limited cell proliferation abilities. Thus, even when the cells are transplanted to a patient, it is difficult to expect long-term cell therapeutic efficacy. However, the umbilical cord blood-derived mononuclear cells retain high differentiation and proliferation abilities and exhibit very low graft-versus-host (GVH) reactions which are side effects caused by transplantation, and thus, can be transplanted to many patients. That is, the umbilical cord blood-derived mononuclear cells are differentiated and proliferated after transplantation, and thus, the proliferation of cervical cancer cells is continuously prevented and immune rejection reactions in vivo hardly occur.

The umbilical cord blood-derived mononuclear cells can be isolated by a known method, e.g., a modified Ficoll-Hypaque method, a 3% gelatin method, or a Ficoll-Hypaque method (Kim et al., Optimal umbilical cord blood processing: Basic study for the establishment of cord blood bank, Korean Journal of Hematopoietic Stem Cell Transplantation. 2000. 5:61-68). In addition, after adding an anticoagulant to umbilical cord blood, mononuclear cells can be isolated by a Ficoll-Paque density gradient centrifugation method.

The composition of the present invention can be administered parenterally, e.g., in the form of subcutaneous injections. For example, the composition can be directly injected to cancer-forming tissue sites. For parenteral administration (e.g., in the form of injections), the composition of the present invention may be formulated into dispersions and/or solutions including a pharmaceutically acceptable carrier, e.g., sterilized deionized water, a buffer (about pH 7), or physiological saline. If necessary, the composition of the present invention may include an additive commonly used in the art, e.g., a preservative or a stabilizer.

The composition of the present invention can be administered to adult patients who suffer from cervical cancer at a dosage of 1×10⁷-10×10⁸ cells/kg, preferably 2×10⁷-4×10⁷ cells/kg, more preferably about 2×10⁷ cells/kg (based on an adult patient with an average weight of about 60 kg). However, an adequate dosage can be changed according to the type and conditions of a disease. For normal adult patients, the composition of the present invention can be administered in a unit dosage form including about 2×10⁷ umbilical cord blood-derived mononuclear cells and a pharmaceutically acceptable carrier.

Hereinafter, the present invention will be described more specifically with reference to the following examples. The following examples are only for illustrative purposes and are not intended to limit the scope of the invention.

### Examples

Caski cells (Korean Cell Line Bank, Cat. NO. 21550), which were human cervical cancer cells, were injected into non-obese diabetic severe-combined immunodeficient (NOD-SCID) mice to establish animal models for human cervical cancer. Mononuclear cells isolated from umbilical cord blood were transplanted in vivo into the mice. The size and weight of tumors formed in the subcutaneous tissues of the mice were measured, and the presence of hematopoietic cells in the periphery blood and the spleen of the mice in which the mononuclear cells were determined.

### Example 1

### Culture of Human Cervical Cancer Cells

Caski cells (Korean Cell Line Bank, Cat. NO. 21550), which were cells derived from patients with cervical cancer, were cultured in RPMI (Rosewell Park Memorial Institute, Gibco-BRL, Korea) supplemented with 10% fetal bovine serum (FBS, Jeil Biotech Services), 0.25 M HEPES (N-2-hydroxyethyl-piperazine-N′-2-ethane-sulfonic acid), and 1% penicillin and streptomycin.

### Example 2

### Isolation of Mononuclear Cells from Umbilical Cord Blood

Umbilical cord blood treated with an anticoagulant (heparin) was added to a 50 ml Falcon tube containing 20 ml of a Ficoll-Paque solution (Amersham Biosciences AB, Sweden), and the mixture was then centrifuged at 2000 rpm at room temperature for 20 minutes. A mononuclear cell fraction of the middle layer was collected, diluted with a 2-fold volume of a phosphate buffered saline (PBS), centrifuged at room temperature for five minutes for washing.

The mononuclear cells thus-obtained were stained with an anti-CD34 antibody which is a stem cell-specific antibody, anti-CD3 and anti-CD19 antibodies which are immune cell-specific antibodies, and an anti-CD45 antibody which is an antibody against CD45 which is expressed in whole mononuclear hematopoietic cells, for 30 minutes, and PBS (D-phosphate buffered saline) was then added thereto. The mixture was centrifuged at 1500 rpm for 5 minutes to remove antibodies that did not form an antibody-antigen complex. The mononuclear cells were washed and analyzed by a flow cytometer (FACSvantage, BD, U.S.A.) to determine the presence of stem cells and immune cells in the mononuclear cells.

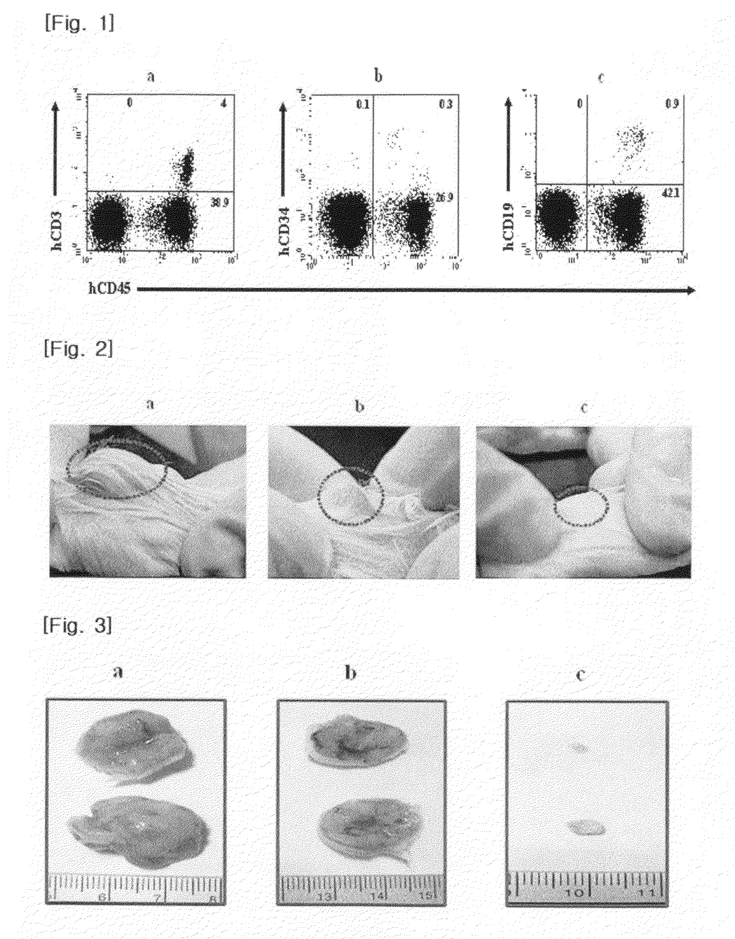

FIG. 1 shows flow cytometry results for stem cells and immune cells in the mononuclear cells derived from the umbilical cord blood, a shows CD3+ T cells, b shows CD34+ hematopoietic stem cells, and c shows CD19+ B cells. As shown in FIG. 1, the mononuclear cells from umbilical cord blood contained CD34+ hematopoietic stem cells, CD3+ T cells and CD19+ B cells.

### Example 3

### Establishment of Experimental Animal Models and In Vivo Transplantation of Mononuclear Cells Derived from Umbilical Cord Blood

NOD-SCID mice (6-8 weeks old) were divided into three groups of 5 mice each.

For the first group, the Caski cells obtained in Example 1 (2×10⁶ cells/mouse) in physiological saline were transplanted into the subcutaneous tissues of the NOD-SCID mice using a 1 ml syringe. For the second group, the Caski cells obtained in Example 1 (2×10⁶ cells/mouse) in physiological saline were transplanted into the subcutaneous tissues of the NOD-SCID mice using a 1 ml syringe, and incubated for about two-three weeks to form cervical tumors. Then, the mononuclear cells obtained in Example 2 (2×10⁷ cells/mouse) were transplanted into the tumor sites of the mice in the same manner as above. For the third group, the Caski cells (2×10⁶ cells/mouse) and the mononuclear cells obtained in Example 2 (2×10⁷ cells/mouse) were transplanted into the subcutaneous tissues of the NOD-SCID mice on the same day in the same manner as above.

### Example 4

### Evaluation of Tumor Suppression Effect of Mononuclear Cells Derived from Umbilical Cord Blood

For the mice of each group treated according to Example 3, the size of tumors was measured using vernier calipers from 6 weeks after the transplantation of the cervical cancer cells. At 8 weeks after the transplantation, 100 μl of peripheral blood was extracted from each of the mice, and the presence of umbilical cord blood-derived T and B cells and myeloid cells in the umbilical cord blood-transplanted mice was analyzed by flow cytometry. The mice were sacrificed, and tumors were excised and weighed (see FIG. 2 through 5).

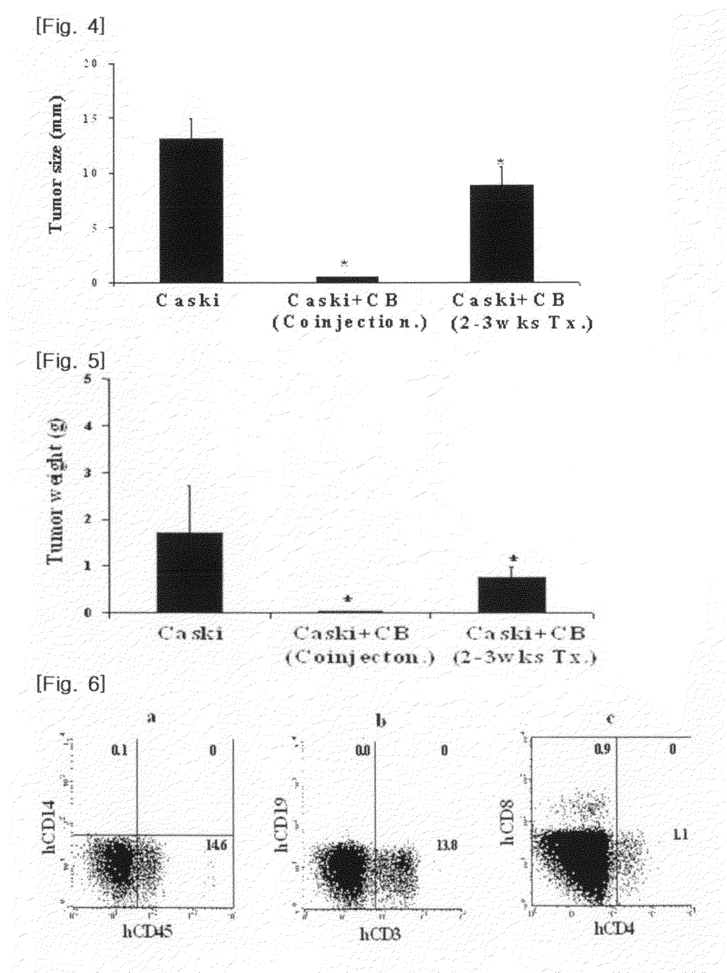

FIG. 2 shows cervical tumors formed in the mice of the first through third groups, and FIG. 3 shows tumors extracted from the mice of the first through third groups. In FIGS. 2 and 3, a shows cervical tumors of the first group (the mice transplanted with only the Caski cells), b shows cervical tumors of the second group (the mice transplanted with the umbilical cord blood-derived mononuclear cells 2-3 weeks after tumor induction), and c shows cervical tumors of the third group (the mice co-transplanted with the cervical cancer cells and the mononuclear cells). FIGS. 4 and 5 shows a tumor size (FIG. 4) and a tumor weight (FIG. 5) in the mice of each group after 8 weeks.

Referring to FIG. 2 through 4, the mice co-transplanted with the cervical cancer cells and the umbilical cord blood-derived mononuclear cells exhibited a significantly higher tumor suppression effect compared to the mice transplanted with only the cervical cancer cells. The mice transplanted with the umbilical cord blood-derived mononuclear cells two weeks after tumor induction also exhibited a tumor suppression effect (P<0.05). Referring to FIG. 5, a tumor suppression effect (0.02±0.02) of the mice co-transplanted with the cervical cancer cells and the umbilical cord blood-derived mononuclear cells was much higher than that (1.7±0.24) of the mice transplanted with only the cervical cancer cells. The mice transplanted with the umbilical cord blood-derived mononuclear cells 2-3 weeks after tumor induction also exhibited a tumor suppression effect (0.7±0.24) (P<0.05).

### Example 5

### Determination of Presence of Umbilical Cord Blood-Derived Cells in Mice

In order to determine the presence of umbilical cord blood-derived mononuclear cells in the mice whose the subcutaneous tissues were transplanted with the umbilical cord blood-derived mononuclear cells (the second and third groups), 500 μl of the peripheral blood extracted from the mice before sacrificed was placed in an anticoagulant-containing tube, and mononuclear cells were isolated from the peripheral blood using a Ficoll-Paque solution (Amersham Biosciences AB, Sweden) in the same manner as in Example 2. On the other hand, mononuclear cells were isolated from the spleen tissues of the mice whose the subcutaneous tissues were transplanted with the umbilical cord blood-derived mononuclear cells (the second and third groups) using a Ficoll-Paque solution (Amersham Biosciences AB, Sweden) in the same manner as in Example 2.

The mononuclear cells were stained with an anti-human CD45 antibody which is a marker specific to human mononuclear hematopoietic cells, and with antibodies specific to human immune cells (B cells, NK cells, and T cells), and the presence of human umbilical cord blood-derived hematopoietic and immune cells in animal models was determined by flow cytometry (see FIG. 6).

FIG. 6 shows umbilical cord blood-derived hematopoietic and immune cells present in the peripheral blood (a, b) and the spleen tissues (c) of the animal models (the mice transplanted with the human cervical cancer cells). Referring to FIG. 6, the CD45+ cells, which are human hematopoietic cells, and the CD3+ cells, which are human T cells, are observed in the mononuclear cells derived from the peripheral blood (a, b), and the CD4+ cells, which are human umbilical cord blood-derived helper T cells, and the CD8+ cells, which are cytotoxic T cells, are observed in the mononuclear cells derived from the spleen tissues.

### INDUSTRIAL APPLICABILITY

According to the present invention, when mononuclear cells derived from umbilical cord blood are transplanted in vivo, cervical cancer can be effectively treated. In particular, the umbilical cord blood-derived mononuclear cells retain high differentiation and proliferation abilities and exhibit very low graft-versus-host (GVH) reactions which are side effects caused by transplantation, and thus, can be transplanted to many patients. That is, the umbilical cord blood-derived mononuclear cells are differentiated and proliferated after transplantation, and thus, proliferation of cervical cancer cells is continuously prevented and immune rejection reactions in vivo hardly occur.

What is claimed is:

1. A composition for in vivo transplantation for the treatment of human cervical cancer, comprising mononuclear cells derived from umbilical cord blood and a pharmaceutically acceptable carrier.

2. The composition of claim 1, wherein the mononuclear cells derived from the umbilical cord blood comprise CD34+ hematopoietic stem cells, CD3+ T cells and CD19+ B cells.
